(19) United States
(12) Patent Application Publication
(10) Pub. No.: US 2026/0285792 A1
(43) Pub. Date: Sep. 24, 2026

(54) PROCESSES FOR THE ACYLATION OF AN AROMATIC COMPOUND

(71) Applicant: University of Kansas, Lawrence, KS (US)

(72) Inventors: Mark Brandon Shiflett, Lawrence, KS (US); Rajkumar Kore, Lawrence, KS (US); Aaron M. Scurto, Lawrence, KS (US)

(73) Assignee: University of Kansas, Lawrence, KS (US)

(21) Appl. No.: 19/563,125

(22) Filed: Mar. 11, 2026

Related U.S. Application Data
(63) Continuation of application No. 18/042,559, filed on Aug. 20, 2021, now Pat. No. 12,606,511.
(60) Provisional application No. 63/069,332, filed on Aug. 24, 2020.

(51) Int. Cl. C07C 45/46; C07C 49/76; C07D 233/58

(52) U.S. Cl. CPC C07C 45/46; C07D 233/58

(57) ABSTRACT

Processes for acylating an aromatic compound are provided. In embodiments, such a process comprises combining an aromatic compound, an acylating agent, and a catalyst composition under conditions to induce acylation of the aromatic compound with the acylating agent, the catalyst composition comprising components selected from the group consisting of a sulfonic acid of formula R—SO₃H, wherein R is a linear alkyl group substituted with one or more halogen atoms; an ionic liquid and an acid; an acid and a base capable of forming an ionic liquid with the acid; an ionic liquid, an acid, and an aromatic; and an acid, a base capable of forming an ionic liquid with the acid, and an aromatic. The ionic liquid does not comprise a metal halide and the catalyst composition is free of a metal halide and the aromatic, if present in the catalyst composition, is not the aromatic compound being acylated.

### CROSS-REFERENCE TO RELATED APPLICATIONS

The present application is a continuation of U.S. patent application Ser. No. 18/042,559, filed Feb. 22, 2023, which is a US National Stage Application under 35 U.S.C. 371 of International Patent Application No. PCT/US21/46882, filed Aug. 20, 2021, which claims priority to U.S. Provisional Patent Application No. 63/069,332, filed Aug. 24, 2020, the entire contents of which are incorporated herein by reference.

### BACKGROUND

Friedel-Crafts acylation of aromatic compounds is an important reaction in the production of aromatic ketones. These aromatic ketones are largely used as intermediates in the synthesis of pharmaceuticals, naproxen, dextromethorphan, ibuprofen and dyes, fragrances, and agrochemicals. The product 4-isobutylacetophenone obtained from the acylation of isobutylbenzene is largely used as an intermediate for the synthesis of ibuprofen and other pharmaceuticals. The traditional catalysts used to synthesize 4-isobutylacetophenone from acylation of isobutylbenzene are AlCl₃, HF, and zeolites. Due to its low activity, AlCl₃ is used in higher than stoichiometric amounts, results in a larger amount of waste, and is non-regenerable after the reaction. HF is an extremely toxic, volatile, and corrosive chemical, and is also typically used in a higher than stoichiometric amount relative to isobutylbenzene. A solid acid catalyst such as an acidic zeolite was investigated in the reaction but the yield obtained was very low (<6%). Recently, a chloroaluminate ionic liquid (IL)-based catalyst was also investigated in the reaction but these are moisture sensitive materials and difficult to handle.

### SUMMARY

The present disclosure provides processes for the acylation of aromatic compounds, including isobutylbenzene.

Processes for acylating an aromatic compound are provided. In embodiments, such a process comprises combining an aromatic compound, an acylating agent, and a catalyst composition under conditions to induce acylation of the aromatic compound with the acylating agent, the catalyst composition comprising components selected from the group consisting of a sulfonic acid of formula R—SO₃H, wherein R is a linear alkyl group substituted with one or more halogen atoms; an ionic liquid and an acid; an acid and a base capable of forming an ionic liquid with the acid; an ionic liquid, an acid, and an aromatic; and an acid, a base capable of forming an ionic liquid with the acid, and an aromatic. The ionic liquid does not comprise a metal halide and the catalyst composition is free of a metal halide and the aromatic, if present in the catalyst composition, is not the aromatic compound being acylated.

In other embodiments, such a process comprises combining an aromatic compound, an acylating agent, and a catalyst composition under conditions to induce acylation of the aromatic compound with the acylating agent, the catalyst composition comprising a sulfonic acid of formula R—SO₃H, wherein R is a linear alkyl group substituted with one or more halogen atoms, wherein the catalyst composition is free of a metal halide and wherein a mole ratio of the catalyst composition to the acylating agent is no more than 0.5:1.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A-1C show illustrative cations which may be used to form an ionic liquid for use in the present catalyst compositions.

FIG. 1D shows illustrative cations which may be used to form an ionic liquid for use in the present catalyst compositions.

FIG. 1E shows illustrative bases for use in the present catalyst compositions.

FIG. 2 shows illustrative anions which may be used to form an ionic liquid for use in the present catalyst compositions.

FIG. 3 shows illustrative acids for use in the present catalyst compositions.

FIG. 4 shows illustrative aromatics for use in the present catalyst compositions.

FIG. 5 shows the reaction scheme for the acylation of isobutylbenzene with acetyl chloride, which may be carried out using the present catalyst compositions.

### DETAILED DESCRIPTION

The present disclosure provides a process for the acylation of aromatic compounds using certain catalyst compositions. At least some embodiments of the process achieve high conversion values (e.g., >99.9%), high selectivities (e.g., 95%), or both. The present processes are more environmentally friendly as compared to existing processes, e.g., those based on HF. Additional advantages include, but are not limited to, non-toxic and moisture stable catalyst composition, much less catalyst composition required, easy recovery of the catalyst composition, and tunable product selectivity via acidity of the catalyst composition.

The present catalyst compositions are multicomponent ionic systems which are typically liquids near room temperature (e.g., 20 to 25° C.). Components which may be used to form the catalyst compositions include certain ionic liquids; acids; bases; and aromatics. Each of these components are described below, followed by a description of various catalyst compositions formed therefrom.

### Ionic Liquids

Various ionic liquids may be used to form the present catalyst compositions. As used in the present disclosure, “ionic liquid” refers to salts composed of at least one cation and at least one anion and are being used in their fluid state. They are generally in their fluid state at or below a temperature of about 100° C.

Representative examples of ionic liquids suitable for use herein are included among those that are described in sources such as J. Chem. Tech. Biotechnol., 68:351-356 (1997); Chem. Ind., 68:249-263 (1996); J. Phys. Condensed Matter, 5: (supp 34B):899-8106 (1993); Chemical and Engineering News, Mar. 30, 1998, 32-37; J Mater. Chem., 8:2627-2636 (1998); Chem. Rev., 99:2071-2084 (1999); and WO 05/113,702 (and references cited therein), each of which is by this reference incorporated herein for the purpose of the ionic liquids disclosed therein.

Many ionic liquids are formed by reacting a nitrogen-containing heterocyclic ring, preferably a heteroaromatic ring, with an alkylating agent (e.g., an alkyl halide) to form a quaternary salt, and performing ion exchange or other suitable reactions with various Lewis acids or their conjugate bases to form the ionic liquid. Some ionic liquids are formed by reacting N-, P-, and S-compounds with a Bronsted acid to quaternize the heteroatom. Examples of suitable heteroaromatic rings include substituted pyridines, imidazole, substituted imidazole, pyrrole and substituted pyrroles. These rings can be alkylated with virtually any straight, branched or cyclic C₁₋₂₀ alkyl group, but the alkyl groups are preferably C₁₋₁₆ groups. Various trialkylphosphines, thioethers and cyclic and non-cyclic quaternary ammonium salts may also be used for this purpose. Ionic liquids suitable for use herein may also be synthesized by salt metathesis, by an acid-base neutralization reaction, or by quaternizing a selected nitrogen-containing compound. The synthesis of other ionic liquids suitable for use herein is described in U.S. Pat. No. 8,715,521, which is by this reference incorporated in its entirety as a part hereof for all purposes. Ionic liquids may also be obtained commercially from several companies such as Merck (Darmstadt, Germany), BASF (Mount Olive N.J.), Fluka Chemical Corp. (Milwaukee Wis.), and Sigma-Aldrich (St. Louis Mo.), Iolitec-Ionic Liquids Technologies, GmbH (Heilbronn, Germany) and Proionic (Graz, Austria).

Ionic liquids suitable for use herein comprise a cation and an anion. A variety of cations and anions may be used. Either or both of the ions may be fluorinated. However, in embodiments, neither of the ions are fluorinated. The ionic liquid may include more than one type of cation, more than one type of anion, or both. However, the ionic liquid may include a single type of cation and a single type of anion. When the ionic liquid includes a single type of cation and a single type of anion, however, this does not preclude some amount of ion exchange with other ions in the catalyst composition (derived from other components of the catalyst composition).

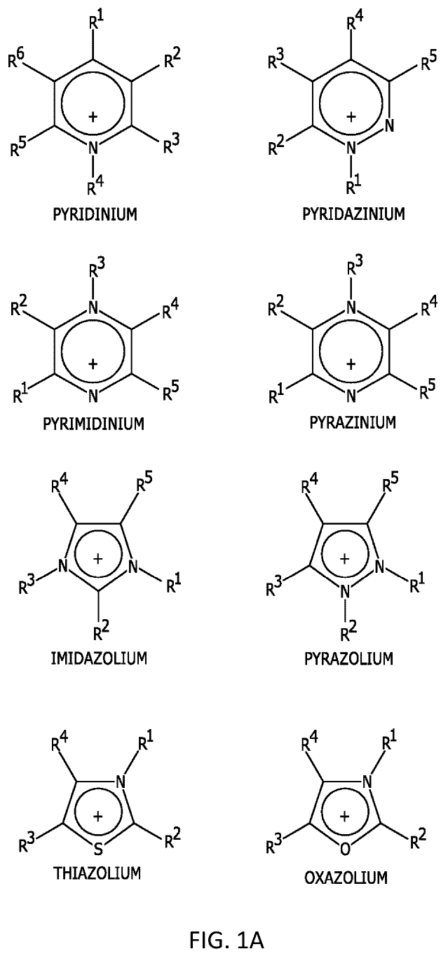

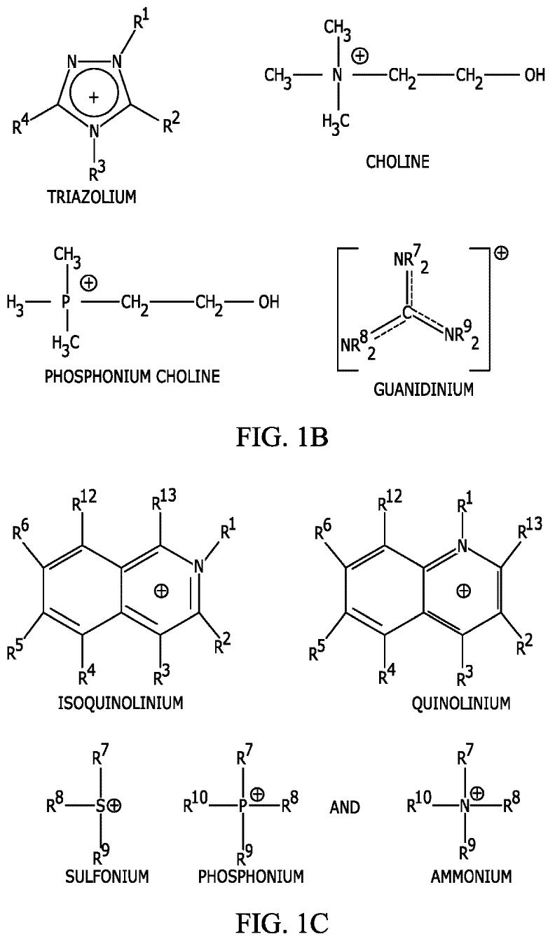

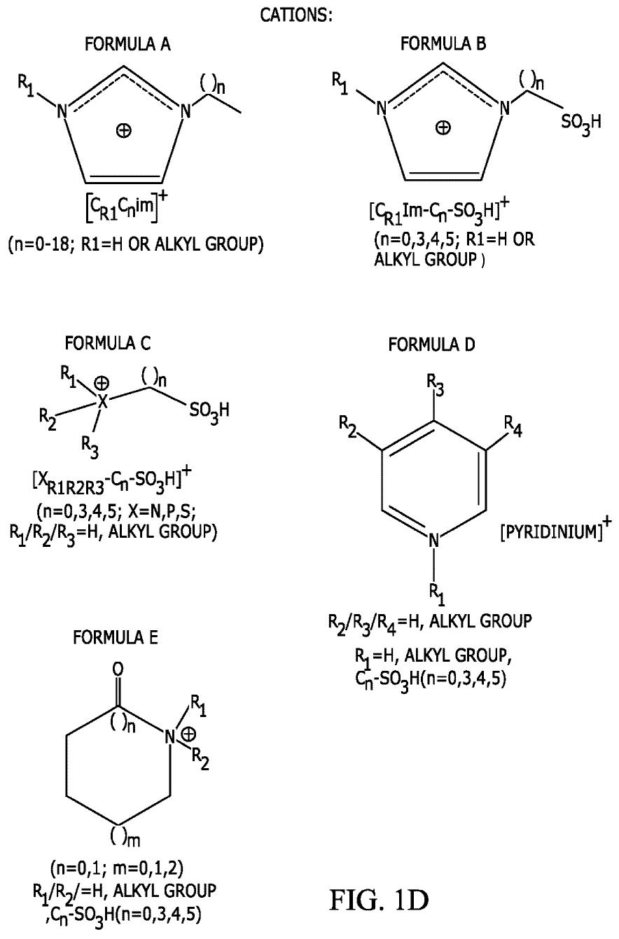

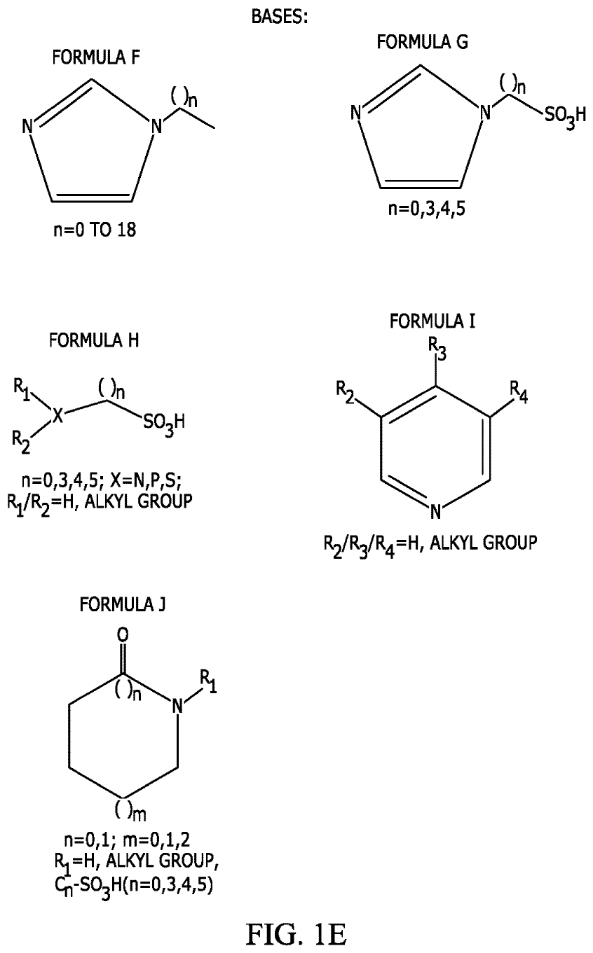

In embodiments, the cation is selected from the group consisting of cations represented by the structures of the formulae shown in FIGS. 1A-1C. In these formulae, the following provisos apply:

(a) R¹, R², R³, R⁴, R⁵, R⁶, R¹² and R¹³ are independently selected from the group consisting of:
(i) H;
(ii) halogen such as F;
(iii) —CH₃, —C₂H₅, or C₃ to C₂₅ straight-chain, branched or cyclic alkane or alkene groups, optionally substituted with at least one member selected from the group consisting of Cl, Br, F, I, OH, NH₂ SH, and SO₃H;
(iv) —CH₃, —C₂H₅, or C₃ to C₂₅ straight-chain, branched or cyclic alkane or alkene groups comprising one to three heteroatoms selected from the group consisting of O, N, Si and S, and optionally substituted with at least one member selected from the group consisting of Cl, Br, F, I, OH, NH₂ and SH;
(v) C₆ to C₂₅ unsubstituted aryl, or C₆ to C₂₅ unsubstituted heteroaryl, groups having one to three heteroatoms independently selected from the group consisting of O, N, Si and S, wherein the unsubstituted aryl or unsubstituted heteroaryl may be bonded to the structure via an alkyl (e.g., —CH₂—) spacer group;
(vi) C₆ to C₂₅ substituted aryl, or C₆ to C₂₅ substituted heteroaryl, groups having one to three heteroatoms independently selected from the group consisting of O, N, Si and S; wherein the substituted aryl or substituted heteroaryl may be bonded to the structure via an alkyl (e.g., —CH₂—) spacer group; and wherein said substituted aryl or substituted heteroaryl has one to three substituents independently selected from the group consisting of:
   (A) —CH₃, —C₂H₅, or C₃ to C₂₅ straight-chain, branched or cyclic alkane or alkene groups, optionally substituted with at least one member selected from the group consisting of Cl, Br, F, I, OH, NH₂ and SH,
   (B) OH,
   (C) NH₂, and
   (D) SH; and
(vii) —(CH₂)ₙSi(CH₂)ₘCH₃, —(CH₂)ₙSi(CH₃)₃, —(CH₂)ₙOSi(CH₃)ₘ, where n is independently 1-4 and m is independently 0-4;

(b) R⁷, R⁸, R⁹, and R¹⁰ are independently selected from the group consisting of: (i) —CH₃, —C₂H₅, or C₃ to C₂₅ straight-chain, branched or cyclic alkane or alkene groups, optionally substituted with at least one member selected from the group consisting of Cl, Br, F, I, OH, NH₂, SH and SO₃H;
(ii) —CH₃, —C₂H₅, or C₃ to C₂₅ straight-chain, branched or cyclic alkane or alkene groups comprising one to three heteroatoms selected from the group consisting of O, N, Si and S, and optionally substituted with at least one member selected from the group consisting of Cl, Br, F, I, OH, NH₂ and SH;
(iii) C₆ to C₂₅ unsubstituted aryl, or C₆ to C₂₅ unsubstituted heteroaryl, groups having one to three heteroatoms independently selected from the group consisting of O, N, Si and S; and
(iv) C₆ to C₂₅ substituted aryl, or C₆ to C₂₅ substituted heteroaryl, groups having one to three heteroatoms independently selected from the group consisting of O, N, Si and S, and wherein the substituted aryl or substituted heteroaryl group has one to three substituents independently selected from the group consisting of:
   (A) —CH₃, —C₂H₅, or C₃ to C₂₅ straight-chain, branched or cyclic alkane or alkene groups, optionally substituted with at least one member selected from the group consisting of Cl, Br, F, I, OH, NH₂ and SH,
   (B) OH,
   (C) NH₂, and
   (D) SH; and
(v) —(CH₂)ₙSi(CH₂)ₘCH₃, —(CH₂)ₙSi(CH₃)₃, —(CH₂)ₙOSi(CH₃)ₘ, where n is independently 1-4 and m is independently 0-4; and

(c) optionally, at least two of R¹, R², R³, R⁴, R⁵, R⁶, R⁷, R⁸, R⁹, and R¹⁰ can together form a cyclic or bicyclic alkyl or alkenyl group.

In embodiments, the ionic liquid comprises a cation selected from one or more members of the group consisting of pyridinium, pyridazinium, pyrimidinium, pyrazinium, imidazolium, pyrazolium, thiazolium, oxazolium, triazolium, phosphonium, ammonium, benzyltrimethylammonium, choline, cholinium, dimethylimidazolium, guanidinium, phosphonium choline, lactam, sulfonium, tetramethylammonium, and tetramethylphosphonium.

In embodiments, the ionic liquid comprises an anion selected from one or more members of the group consisting of: [CH₃CO₂]⁻, [HSO₄]⁻, [CH₃OSO₃]⁻, [C₂H₅OSO₃]⁻, [CH₃C₆H₄SO₃]⁻([TSO]⁻), [CO₃]²⁻, [HCO₃]⁻, [NO₂]⁻, [NO₃]⁻, [SO₄]²⁻, [PO₃]³⁻, [HPO₃]²⁻, [H₂PO₃]¹⁻, [PO₄]³⁻, [HPO₄]²⁻, [H₂PO₄]⁻, [HSO₃]⁻, [CuCl₂]⁻, Cl⁻, Br⁻, I⁻, SCN⁻, carborates optionally substituted with alkyl or substituted alkyl; carboranes optionally substituted with alkylamine, substituted alkylamine, alkyl or substituted alkyl; and a fluorinated anion.

In embodiments, the ionic liquid comprises an anion selected from one or more members of the group consisting of aminoacetate, ascorbate, benzoate, catecholate, citrate, dimethylphosphate, formate, fumarate, glycolate, glyoxylate, iminodiacetate, isobutyrate, kojate, lactate, levulinate, oxalate, pivalate, propionate, pyruvate, salicylate, succinamate, succinate, tiglate, tetrafluoroborate, tetrafluoroethanesulfonate, tropolonate, [CH₃CO₂]⁻, [HSO₄]⁻, [CH₃SO₃]⁻, [CH₃OSO₃]⁻, [C₂H₅OSO₃]⁻, [CH₃C₆H₄SO₃]⁻, [CO₃]²⁻, [HCO₃]⁻, [NO₂]⁻, [NO₃]⁻, [SO₄]²⁻, [PO₃]³⁻, [HPO₄]²⁻, [H₂PO₄]⁻, [HSO₃]⁻, [CuCl₂]⁻, Cl⁻, Br⁻, I⁻, SCN⁻, [BF₄]⁻, [PF₆]⁻, [SbF₆]⁻, [CF₃SO₃]⁻, [HCF₂CF₂SO₃]⁻, [CF₃HFCCF₂SO₃]⁻, [CHF₂CF₂CF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, [(CF₂HCF₂SO₂)₂N]⁻, [(CF₃CFHCF₂SO₂)₂N]⁻, [N(CN)₂]⁻, F⁻, and anions represented by the structure of the following formula, [R₁₁COO]⁻, wherein R¹¹ is selected from the group consisting of:
(i) —CH₃, —C₂H₅, or C₃ to C₁₀ straight-chain, branched or cyclic alkane or alkene groups, optionally substituted with at least one member selected from the group consisting of Cl, Br, F, I, OH, NH₂ and SH;
(ii) —CH₃, —C₂H₅, or C₃ to C₁₀ straight-chain, branched or cyclic alkane or alkene groups that contain one to three heteroatoms selected from the group consisting of O, N, Si and S, and are optionally substituted with at least one member selected from the group consisting of Cl, Br, F, I, OH, NH₂ and SH;
(iii) C₆ to C₁₀ unsubstituted aryl, or C₆ to C₁₀ unsubstituted heteroaryl, groups having one to three heteroatoms independently selected from the group consisting of O, N, Si and S; and
(iv) C₆ to C₁₀ substituted aryl, or C₆ to C₁₀ substituted heteroaryl, groups having one to three heteroatoms independently selected from the group consisting of O, N, Si and S; and wherein the substituted aryl or substituted heteroaryl group has one to three substituents independently selected from the group consisting of:
   (A) —CH₃, —C₂H₅, or C₃ to C₁₀ straight-chain, branched or cyclic alkane or alkene groups, optionally substituted with at least one member selected from the group consisting of Cl, Br, F, I, OH, NH₂ and SH,
   (B) OH,
   (C) NH₂, and
   (D) SH.

In embodiments, the cation of the ionic liquid is selected from an imidazolium, an ammonium, a phosphonium, a sulfonium, a pyridinium, and a lactam. The cation may be protic or aprotic. The proton in the protic cation may be from a —SO₃H group. Illustrative imidazolium, ammonium, phosphonium, sulfonium, pyridinium, and lactam cations are shown in FIG. 1D. In embodiments, the cation of the ionic liquid is selected from the group consisting of cations represented by the structures of the formulae shown in FIG. 1D, i.e., Formulae A-E. In these formulae, the provisos noted in FIG. 1D apply.

In embodiments, the cation of the ionic liquid is an imidazolium having Formula A or B shown in FIG. 1D, an imidazolium having the formula shown in FIG. 1A, or an ammonium having the formula shown in FIG. 1C. In these formulae, the provisos noted in FIG. 1D and FIGS. 1A,1C apply, respectively.

The anion of the ionic liquid may be a sulfonate. The sulfonate may have the formula [R—SO₃]⁻, wherein R is an alkyl group or an aryl group. The alkyl group may be a linear alkyl group in which the number of carbons may range from, e.g., 1 to 12. The alkyl group may be unsubstituted, by which it is meant the alkyl group contains only carbon and hydrogen and no heteroatoms. The alkyl group may be substituted, by which it is meant an unsubstituted alkyl group in which one or more bonds to a carbon(s) or hydrogen(s) are replaced by a bond to non-hydrogen and non-carbon atoms. Non-hydrogen and non-carbon atoms include, e.g., a halogen atom such as F. Aryl groups may be unsubstituted or substituted as described above with respect to alkyl groups. However, substituted aryl groups also refer to an unsubstituted monocyclic aryl group in which one or more carbon atoms are bonded to an alkane. The alkane may be linear, have various numbers of carbon, and may be unsubstituted or substituted as described above with respect to alkyl groups.

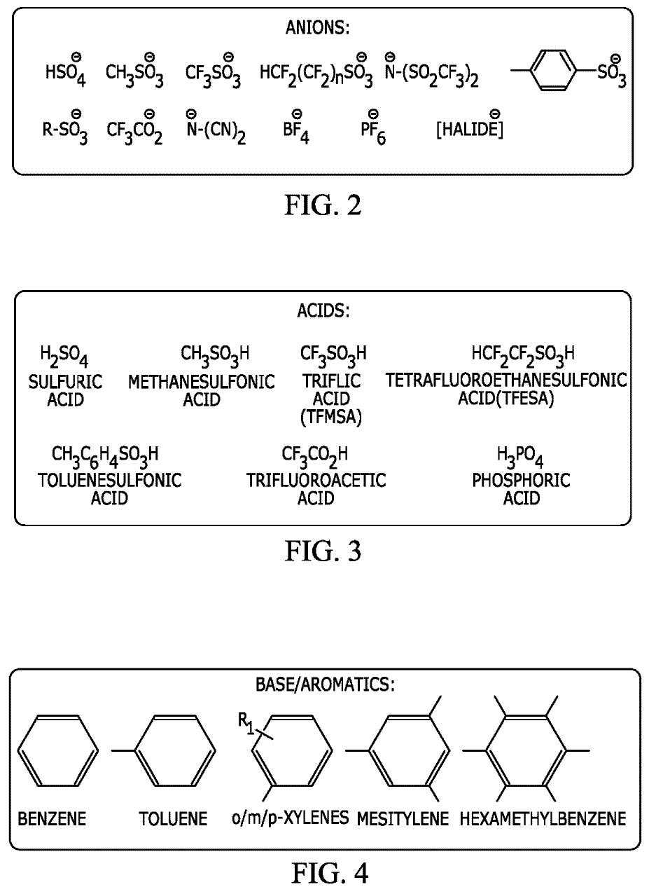

The anion may be a carboxylate. The carboxylate may have the formula [R−CO₂]⁻, wherein R is an alkyl group as described above with respect to sulfonate. This means that fluoroalkane carboxylates are encompassed, e.g., R may be CF₃, HCF₂CF₂, CF₃HFCCF₂, etc. The carboxylate (or fluoroalkane carboxylate) may be a dicarboxylate, a tricarboxylate, a tetracarboxylate, etc. Other anions which may be used include [HSO₄]⁻, dicyanamide; and inorganic anions such as [BF₄]⁻, [PF₆]⁻, and a halide. Illustrative anions are shown in FIG. 2. In [HCF₂(CF₂)ₙSO₃]⁻, n may be 0, 1, 2, or 3.

In embodiments, the anion of the ionic liquid is [HSO₄]⁻ or [HCF₂CF₂SO₃]⁻.

Ionic liquids disclosed in the following references may also be used: U.S. Pat. Nos. 8,771,626; 8,779,220; 8,808,659; U.S. Pat. Pub. No. 20100331599; U.S. Pat. Nos. 7,432,408; 9,914,674; U.S. Pat. Pub. No. 20160289138; U.S. Pat. Pub. No. 20140113804; U.S. Pat. Pub. No. 20160167034; U.S. Pat. Pub. No. 20150315095; and U.S. Pat. Nos. 9,567,273; 9,346,042; 9,260,668; 9,096,487; 8,692,048; 8,653,318; 8,633,346; 8,569,561; 8,552,243; and 7,285,698. Each of these is by this reference incorporated herein for the purpose of the ionic liquids disclosed therein.

In the ionic liquids, various relative amounts of the cation(s) and anion(s) may be used. In embodiments, the molar ratio of the cation:anion is in the range of from 1:1 to 4:1.

Illustrative specific ionic liquids are also provided in the Examples, below.

In embodiments, the following provisos apply: the ionic liquid is not a haloaluminate (e.g., chloroaluminate), a halozincate, a haloferrate, a halogallate, a halostannate, a haloindate, a halochromate, a halocuprate, a halotitannate, a halozirconate, or a halopalladate and the ionic liquid is not formed from a metal halide (thus, neither the cation(s) nor the anion(s) of the ionic liquid are derived from such a metal halide). In such embodiments, the catalyst composition is free of such ionic liquids and free of a metal halide. In embodiments, the ionic liquid is not formed from a non-metal halide (thus, neither the cation(s) nor the anion(s) of the ionic liquid are derived from such a non-metal halide). In such embodiments, the catalyst composition is free of such ionic liquids and free of a non-metal halide. The term “halide” may be used to refer to both metal and non-metal halides. The embodiments in this paragraph do not preclude the use of certain halogenated cation(s), anion(s) (e.g., HCF₂CF₂SO₃⁻), halogenated acids (e.g., haloalkane sulfonic acids), halogenated bases, and halogenated aromatic compounds (e.g., halogenated benzene). Throughout the present disclosure, the term “free” means that the amount of the relevant component is zero or sufficiently close to zero to have no material effect on the properties of the catalyst composition.

Known methods may be used to prepare ionic liquids. Other ionic liquids may be commercially available. Illustrative methods for synthesizing ionic liquids are described in the Examples, below.

### Acids

Various acids may be used to form the present catalyst compositions, including combinations of different types of acids. However, a single type of acid may also be used. Mineral acids may be used, e.g., sulfuric acid, phosphoric acid, hydrofluoric acid, hydrochloric acid.

Sulfonic acids may be used. The sulfonic acid may have the formula R—SO₃H, wherein R is an alkyl group or an aryl group. The alkyl group may be linear, branched, or cyclic and may have a number of carbons in a range from, e.g., 1 to 12. The alkyl group may be unsubstituted, by which it is meant the alkyl group contains only carbon and hydrogen and no heteroatoms. The alkyl group may be substituted, by which it is meant an unsubstituted alkyl group in which one or more bonds to a carbon(s) or hydrogen(s) are replaced by a bond to non-hydrogen and non-carbon atoms. Non-hydrogen and non-carbon atoms include, e.g., a halogen atom such as F, Cl, Br, and I. Aryl groups may be unsubstituted or substituted as described above with respect to alkyl groups. However, substituted aryl groups also refer to an unsubstituted monocyclic aryl group in which one or more carbon atoms are bonded to an alkane. The alkane may be linear, branched, or cyclic, have various numbers of carbon atoms, and may be unsubstituted or substituted as described above with respect to alkyl groups.

Carboxylic acids may be used. The carboxylic acid may have the formula R—CO₂H, wherein R is an alkyl or an aryl group as described above with respect to sulfonic acid.

Illustrative specific acids are shown in FIG. 3. In embodiments, the acid is triflic acid. In embodiments, the acid is tetrafluoroethanesulfonic acid.

In embodiments, the acid is not hydrofluoric acid and the catalyst composition is free of hydrofluoric acid.

### Bases

In embodiments, a base is used which forms, in situ, an ionic liquid when combined with any of the disclosed acids in forming the catalyst composition. Thus, any base which generates any of the cations described in “Ionic Liquids,” above, upon combination with any of the disclosed acids may be used. By way of illustration, the base may be an imidazole, an ammonia, a phosphine, a sulfide, a pyridine, or a lactam. The base be selected from the group of compounds having any of the formulae shown in FIG. 1E, i.e., Formulae F-J. In these formulae, the alkyl group may be a linear alkyl group in which the number of carbons may range from, e.g., 1 to 12. The alkyl group may be unsubstituted, by which it is meant the alkyl group contains only carbon and hydrogen and no heteroatoms. The alkyl group may be substituted, by which it is meant an unsubstituted alkyl group in which one or more bonds to a carbon(s) or hydrogen(s) are replaced by a bond to non-hydrogen and non-carbon atoms. Non-hydrogen and non-carbon atoms include, e.g., a halogen atom such as F.

In embodiments, the base is an imidazole, e.g., having Formula F or Formula G in FIG. 1E In embodiments, the base is an ammonia.

The catalyst composition may be formed from different types of bases. However, a single type of base may also be used.

### Aromatics

Various aromatics may be used to form the present catalyst compositions, including combinations of different types of aromatics. However, a single type of aromatic may also be used.

The aromatic may be monocyclic having one or more unfused aromatic rings. Each aromatic ring may have various members, e.g., a 5-membered ring, a six-membered ring, etc. Monocyclic aromatics may be unsubstituted, by which it is meant the aromatic contains only carbon and hydrogen and no heteroatoms. Unsubstituted monocyclic aromatics have a single aromatic ring. Monocyclic aromatics may be substituted, by which it is meant an unsubstituted aromatic in which one or more bonds to a carbon(s) or hydrogen(s) are replaced by a bond to non-hydrogen and non-carbon atoms. Non-hydrogen and non-carbon atoms include, e.g., a halogen atom such as F, Cl, Br; O; N; etc. However, substituted monocyclic aromatics also refer to an unsubstituted monocyclic aromatic in which one or more carbon atoms are bonded to an unsubstituted or substituted alkane or another unsubstituted or substituted monocyclic aromatic. The alkane may be linear or branched, have various numbers of carbon atoms, and may be unsubstituted or substituted as described above with respect to the definition of alkyl groups in “Acids.” Thus, monocyclic aromatics include benzene, biphenyl, triphenyl, furan, pyridine, pyrrole, etc. (each which may be unsubstituted or substituted).

The monocyclic aromatic may have the formula C₆R₆, wherein each R is independently selected from hydrogen, a halogen, and an alkyl group. The alkyl group may be linear or branched have various numbers of carbon atoms and may be unsubstituted or substituted as described above with respect to the definition of alkyl groups in “Acids.” Illustrative such monocyclic aromatics are shown in FIG. 4.

Polycyclic aromatics may be used. Polycyclic aromatics have fused aromatic rings (e.g., two, three, etc. rings). Each ring may have various members and may be unsubstituted or substituted as described for monocyclic aromatics. Naphthalene, anthracene, phenanthrene, benzofuran are illustrative polycyclic aromatics.

In embodiments, the aromatic is hexamethylbenzene.

Similar to the bases described above, it is noted that the aromatic used in the catalyst composition may be one which forms, in situ, an ionic liquid when combined with the acid to form the catalyst composition.

### Catalyst Compositions

One or more of any of the disclosed ionic liquids, acids, bases, and aromatics may be used to form the present catalyst compositions. As noted above, ion exchange generally occurs between the various components of the catalyst compositions, once formed. In addition, there may be some overlap between compounds suitable for the various components, e.g., some compounds may be suitable as a base and an aromatic. However, catalyst compositions described as comprising, e.g., an “ionic liquid,” an “acid”, and an “aromatic” refer to catalyst compositions in which separate and distinct chemicals have been combined to form the catalyst composition regardless of how the various ions may subsequently rearrange/associate therein. For example, a catalyst composition described as comprising an “ionic liquid,” an “acid”, and an “aromatic” means that a chemically distinct ionic liquid, a chemically distinct acid, and a chemically distinct aromatic were combined to form the catalyst composition. As another example, a catalyst composition described as comprising an ionic liquid and an acid refers to compositions in which a chemically distinct ionic liquid and a chemically distinct acid were combined to form the catalyst composition.

The particular component or combination of components may be selected to achieve certain behavior, e.g., desired conversion or desired product selectivity. Specific combinations are illustrated in the Examples below, but these are not intended to be limiting. Similarly, for catalyst compositions comprising more than one component, the components may be present at various amounts selected to achieve certain behavior. By way of illustration, as shown in the Examples, below, it has been found that both conversion and product selectivity are particularly sensitive to the type and amount of the acid component used. (See Table 2.)

Table 1, below, lists catalyst compositions which may be used. The various components may be selected from those described above. More than one type of each component may be used, i.e., more than one type of ionic liquid/base, more than one type of acid, and/or more than one type of aromatic. In other such embodiments, a single type of each component may be used. The provisos described above may apply, e.g., no metal halides; no hydrofluoric acid. The parameters x and y refer to weight percents, as further described below.

TABLE 1
Catalyst Compositions.

[IL]ₓ-[Acid]₍₁₀₀₋ₓ₎
[IL]ₓ-[Acid]₍₁₀₀₋ₓ₎-[Aromatic]_{y}
[Base]ₓ-[Acid]₍₁₀₀₋ₓ₎
[Base]ₓ-[Acid]₍₁₀₀₋ₓ₎-[Aromatic]_{y}

In the catalyst compositions [IL]ₓ-[Acid]₁₀₀₋ₓ and [Base]ₓ-[Acid](₁₀₀₋ₓ), the parameter x refers to a weight (wt) %, i.e., ((weight of the ionic liquid/base)/(combined weight of the ionic liquid/base and the acid))*100. In embodiments, x is in a range of from 0.5 wt % to 90 wt % and the acid is present at an amount in a range of from 99.5 wt % to 10 wt %. In embodiments, x is 0 and only the acid is present. This includes embodiments in which the ionic liquid/base is present at an amount in a range of from 2 wt % to 80 wt %, from 5 wt % to 60 wt %, from 5 wt % to 30 wt % or from 5 wt % to 20 wt % and the acid is present at an amount in a range of from 98 wt % to 20 wt %, from 95 wt % to 40 wt %, from 95 wt % to 70 wt % or from 95 wt % to 80 wt %, respectively.

In the catalyst compositions [IL]ₓ-[Acid](₁₀₀₋ₓ)-[Aromatic]_{y} and [Base]ₓ-[Acid](₁₀₀₋ₓ)-[Aromatic]_{y}, x is as defined above and y refers to ((weight of the aromatic)/(combined weight of the ionic liquid/base, and acid))*100. In embodiments, the aromatic component may be present in any amount up to its saturation point in the composition. In embodiments, y is in a range of from of 0.1 wt % to 25 wt %. This includes from 1 wt % to 15 wt %, 1 wt % to 10 wt %, from 3 wt % to 9 wt %, or from 5 wt % to 8 wt %. In embodiments, y may be in a range of from 0.1 wt % to 100 wt % or from 0.1 wt % to 50 wt %.

An amount of water may be present in the catalyst composition. However, in embodiments, the catalyst composition consists or consists essentially of the components of Table 1.

Specific, illustrative catalyst compositions are provided in the Examples, below. As shown in the Examples, in embodiments, a sulfonic acid may be used as a catalyst composition. Any of the sulfonic acids described above may be used. This includes sulfonic acids having the formula R—SO₃H, wherein R is a linear alkyl group substituted with one or more halogen atoms. In embodiments, the halogen atom is F. The number of carbon atoms in the linear alkyl group may be from 1 to 12, 1 to 10, 1 to 8, 1 to 6 or 1 to 4.

Other components may be included in the catalyst compositions such as multi-ammonium salts/surfactants described in R. Kore, B. Satpati, R. Srivastava, Synthesis of Dicationic Ionic Liquids and their Application in the Preparation of Hierarchical Zeolite Beta, Chemistry—A European Journal, 17 (2011) 14360-14365 and R. Kore, R. Srivastava, B. Satpati, ZSM-5 zeolite nanosheets with remarkably improved catalytic activity synthesized using a new class of structure directing agents, Chemistry—A European Journal, 20 (2014) 11511-11521, both of which are hereby incorporated by reference in their entirety.

The present catalyst compositions may be made by combining the desired components (together or sequentially) at the desired relative amounts. The synthesis may be carried out while stirring and under room temperature. Other details are provided in the Examples, below.

With regards to the present catalyst compositions comprising three components, an acid, an aromatic, and either an ionic liquid or a base which forms, in situ, an ionic liquid with the acid, the following is noted. Without wishing to be bound to any particular theory, it is believed that the three components (or ions generated from the three components) may associate to form a molecular complex having unique, synergistic properties, as distinguished from a simple mixture of the individual components. In the present disclosure, terms such as “ternary complex,” “clathrate,” and the like may be used to describe this molecular complex. However, such terms are not intended to limit the scope of structural form of the molecular complex or catalyst composition. The term “ternary mixture” may also be used in reference to the catalyst composition. Catalyst compositions comprising two components, e.g., an acid and an ionic liquid may be referred to as “binary mixtures.”

### Acylation Process

The present catalyst compositions may be used in a process to acylate an aromatic compound. The phrase “aromatic compound” is used to distinguish an aromatic that may be present in the catalyst compositions. It is noted that the aromatic compound to be acylated may itself form a ternary complex with an acid and an ionic liquid/base in a catalyst composition used for the acylation. In embodiments, a catalyst composition is used for the alkylation which comprises any of the disclosed acids, an aromatic, and an ionic liquid or a base, the aromatic and the base, if present, are distinct chemical entities from the aromatic compound to be acylated. This means that either the aromatic/base are different chemical compounds from the aromatic compound to be acylated (i.e., are not the same chemical compound) or are the same chemical compound, but included separately at a separate amount in the catalyst composition.

In embodiments, a process to acylate an aromatic compound comprises combining an aromatic compound, an acylating agent, and any of the disclosed catalyst compositions under conditions to induce acylation of the aromatic compound. Under the appropriate conditions, the present catalyst compositions can catalyze the addition of an acyl group from the acylating agent to the aromatic compound (i.e., induce acylation). As further described below, the aromatic compound may be substituted and the acyl group may be added to the aromatic compound at its para position. The process may further comprise recovering the acylated aromatic compound from the reaction mixture.

The aromatic compound can have formula:

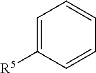

wherein R⁵ is H, halogen, hydroxyl, substituted or unsubstituted C₁-C₈ alkyl, or substituted or unsubstituted C₁-C₈ cycloalkyl. In embodiments, R⁵ is substituted or unsubstituted C₁-C₈ alkyl. In embodiments, R⁵ is unsubstituted C₁-C₈ alkyl. In embodiments, R⁵ is unsubstituted C₄-C₆ alkyl. In embodiments, R⁵ is isobutyl. In embodiments, the aromatic compound is isobutylbenzene. Unsubstituted alkyl/cycloalkyl means the group contains only carbon and hydrogen and no heteroatoms. Substituted alkyl/cycloalkyl refers to the unsubstituted group in which one or more bonds to a carbon(s) or hydrogen(s) are replaced by a bond to non-hydrogen and non-carbon atoms. Non-hydrogen and non-carbon atoms include, e.g., a halogen atom such as F.

The acylating agent can be an acyl halide of formula:

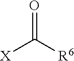

wherein X is a halogen; and R⁶ is H, halogen, hydroxyl, substituted or unsubstituted C₁-C₈ alkyl, substituted or unsubstituted C₁-C₈ cycloalkyl, or substituted or unsubstituted aryl. Unsubstituted and substituted alkyl/cycloalkyl have been defined above with respect to the aromatic compound. Aryl groups may be monocyclic or polycyclic as described in “Aromatics” above and may be unsubstituted or substituted as described with respect to alkyl/cycloalkyl groups. However, substituted aryl groups also refer to an unsubstituted monocyclic aryl group in which one or more carbon atoms are bonded to an alkane. The alkane may be linear, have various numbers of carbon, and may be unsubstituted or substituted as described with respect to alkyl/cycloalkyl groups.

In embodiments, X is chloride. In embodiments, R⁶ is substituted or unsubstituted C₁-C₈ alkyl. In embodiments, R⁶ is unsubstituted C₁-C₈ alkyl. In embodiments, R⁶ is unsubstituted C₁-C₄ alkyl. In embodiments, R⁶ is methyl. In embodiments, the acylating agent is acetyl chloride.

The acylating agent can be an acid anhydride of formula:

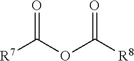

wherein R⁷ and Rare independently H, halogen, hydroxyl, substituted or unsubstituted C₁-C₈ alkyl, substituted or unsubstituted C₁-C₈ cycloalkyl, substituted or unsubstituted aryl, or wherein, as valence permits, R⁷ and R⁸, together with the atoms to which they are attached, form a 4-10 membered cyclic moiety. Unsubstituted and substituted alkyl/cycloalkyl have been defined above with respect to the aromatic compound and unsubstituted and substituted aryl groups have been defined above with respect to the acyl halide.

In embodiments, R⁷ and R⁸ are independently H, halogen, hydroxyl, or substituted or unsubstituted C₁-C₈ alkyl. In embodiments, R⁷ and R⁸ are independently substituted or unsubstituted C₁-C₈ alkyl. In embodiments, R⁷ and R⁸ are independently unsubstituted C₁-C₈ alkyl. In embodiments, R⁷ and R⁸ are independently unsubstituted C₁-C₄ alkyl. In embodiments, the acylating agent is acetic anhydride.

Combinations of different types of aromatic compounds and combinations of different types of acylating agents may be used in the process.

The conditions under which acylation is induced refer to parameters such as the amount of the catalyst composition, the amount of the acylating agent; the amount of the aromatic compound; the reaction temperature; and the reaction time. These parameters may be adjusted to provide, e.g., a desired conversion and/or desired product selectivity. Illustrative values of these parameters are provided in the Examples below. Of course, the values may be scaled up as necessary for commercial processes. It is noted that due to the activity of the present catalytic compositions, generally much less of the catalytic composition is required as compared to existing processes. In embodiments, the mole ratio of the catalytic composition (total moles thereof) as compared to the acylating agent compound (total moles thereof) to be acylated is in a range of from 0.01:1 to 0.5:1. This includes from 0.01:1 to 0.2:1 and from 0.01:1 to 0.1:1. In embodiments, a mole ratio of the catalyst composition to the acylating agent is no more than 0.5:1. This includes no more than 0.2:1 and no more than 0.1:1. In other embodiments, however, a mole ratio of the catalyst composition to the acylating agent is up to 1:1 or 2:1.

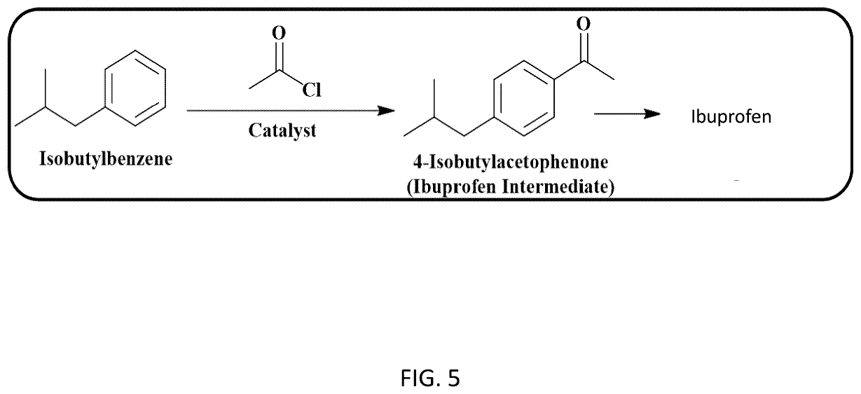

A variety of reactor systems may be used to carry out the present processes, including batch, semi-continuous, continuous, and spray reactor systems. An illustrative process showing the acylation of isobutylbenzene with acetyl chloride is shown in FIG. 5.

The present catalyst compositions and acylation processes may be characterized as being capable of achieving certain properties or results, including a percent conversion and a percent selectivity (for a particular product). Known methods may be used to calculate these values. In embodiments, the conversion is at least 90%, at least 95%, at least 99% or at least 99.9%. In embodiments, the selectivity for adding an acyl group of an acylating agent to a substituted aromatic compound at its para position is at least 85%, at least 90%, at least 95%, or at least 99%. These properties may be referenced with respect to a particular set of reaction conditions, e.g., a set of reaction conditions as set forth in the Examples, below.

### EXAMPLES

### Example 1. Preparation of Ionic Liquids

### Example 1—I: Preparation of N-methyl imidazolium hydrogen sulfate [C₁im][HSO₄]IL

In a 50 mL round bottom flask, equipped with a stir bar, an amount of N-methyl imidazole (4.11 g, 0.05 mol) was placed and then liquid H₂SO₄ acid (4.95 g, 0.05 mol) was added dropwise at 5° C. After addition, the reaction mixture was stirred at 80° C. for 4 h, giving a liquid ionic liquid [C₁im][HSO₄].

### Example 1—II: Preparation of N-methyl, N-sulfonic acid imidazolium tetrafluoroethane sulfonate [C₁im-SO₃H][TFES] IL

In a 500 mL round bottom flask, equipped with a stir bar, N-methyl imidazole (8.21 g, 0.10 mol) was reacted with chlorosulfonic acid (12.20 g, 0.104 mol) in dry dichloromethane solvent (40 mL). After addition, the reaction mixture was stirred for 12 h. The dichloromethane solvent was removed under reduced pressure, yielding a white solid. The solid was washed twice with dry diethyl ether and dried under vacuum, yielding a white solid of [C₁im-SO₃] zwitterion type of salt. For the synthesis of [C₁im-SO₃H][TFES] ionic liquid, a solid [C₁im-SO₃] zwitterionic salt (8.10 g, 0.05 mol) was placed in a 20 mL screw top borosilicate glass vial and tetrafluoroethanesulfonic acid (TFESA) (9.15 g, 0.05 mol) was added dropwise. After addition of the reactants, the reaction mixture was stirred at room temperature for 4 h, giving a brown liquid IL [C₁im-SO₃H][TFES].

### Example 2. Preparation of Binary Mixtures of an Ionic Liquid and an Acid

### Example 2.1—I: Preparation of ([C₁im-SO₃H][TFES])₁₀-(TFMSA)₉₀ IL

In a 40 mL glass tube, equipped with a stir bar, TFMSA (0.45 g) and ionic liquid [C₁im-SO₃H][TFES] (0.05 g) was added at 10:90 wt % and mixed by handshake. The reaction mixture was stirred at room temperature for 5 min, giving a liquid double salt ionic liquid ([C₁im-SO₃H][TFES])₁₀-(TFMSA)₉₀.

### Example 2.1—II. Preparation of ([C₁im-SO₃H][TFES])₂₀-(TFMSA)₈₀ IL

In a 40 mL glass tube, equipped with a stir bar, TFMSA (0.40 g) and ionic liquid [C₁im-SO₃H][TFES] (0.10 g) was added at 20:80 wt % and mixed by handshake. The reaction mixture was stirred at room temperature for 5 min, giving a liquid double salt ionic liquid ([C₁im-SO₃H][TFES])₂₀-(TFMSA)₈₀.

### Example 2.1—III: Preparation of ([C₁im-SO₃H][TFES])₅₀-(TFMSA)₅₀ IL

In a 40 mL glass tube, equipped with a stir bar, TFMSA (0.25 g) and ionic liquid [C₁im-SO₃H][TFES] (0.25 g) was added at 50:50 wt % and mixed by handshake. The reaction mixture was stirred at room temperature for 5 min, giving a liquid double salt ionic liquid ([C₁im-SO₃H][TFES])₅₀-(TFMSA)₅₀.

### Example 2.2—I: Preparation of ([C₁C₄im][HSO₄])₁₀—(H₂SO₄)₉₀ IL

In a 40 mL glass tube, equipped with a stir bar, H₂SO₄ (0.45 g) and IL [C₁C₄im][HSO₄] (0.05 g, commercially available) was added at 10:90 wt % and mixed by handshake. The reaction mixture was stirred at room temperature for 5 min, giving a liquid double salt ionic liquid ([C₁C₄im][HSO₄])₁₀—(H₂SO₄)₉₀.

### Example 2.3—I Preparation of ([N₁₁₁₍₁₆₎][HSO₄])₁₀-(TFMSA)₉₀IL

In a 40 mL glass tube, equipped with a stir bar, TFMSA (0.45 g) and ionic liquid [N₁₁₁₍₁₆₎][HSO₄] (0.05 g, commercially available) was added at 10:90 wt % and mixed by handshake. The reaction mixture was stirred at room temperature for 5 min, giving a liquid double salt ionic liquid ([N₁₁₁₍₁₆₎][HSO₄])₁₀-(TFMSA)₉₀.

### Example 3. Preparation of Ternary Mixtures of an Ionic Liquid, an Acid, and an Aromatic

### Example 3.1—I: Preparation of ([C₁im—SO₃H][TFES])₁₀-(TFMSA)₉₀-(HMB)₁₀ Clathrate

In a 40 mL glass tube, equipped with a stir bar, TFMSA (0.45 g) and ionic liquid [C₁im-SO₃H][TFES] (0.05 g) were added at 10:90 wt % and mixed by handshake. After a minute, 10 wt % of hexamethylbenzene (HMB; 0.05 g) was added and the reaction mixture was stirred at room temperature for 5 min, giving a liquid double salt ionic liquid clathrate ([C₁im-SO₃H][TFES])₁₀-(TFESA)₉₀-(HMB)₁₀.

### Example 4. Acylation of Isobutylbenzene Reaction Apparatus and Procedure

Friedel-Crafts acylation of isobutylbenzene experiments were performed in a 20 mL glass reactor. The reaction mixture was analyzed offline by gas chromatography (GC), equipped with a flame ionization detector, and a DB-5 100 m column (J&W Scientific). Helium was used as the GC carrier gas and as the flame ionization detector (FID) makeup gas. The analysis conditions were: split ratio=50:1, injector temperature=280° C., detector temperature=300° C., carrier gas flow rate=20 sccm. The temperature program for GC analysis was as follows: initial column temperature 150° C./hold for 1 min, 10° C./min to 300° C./hold for 5 min.

All experiments were performed in batch. A typical experiment began with the addition of the catalyst into the 20 mL glass reactor equipped with a Teflon coated magnetic stir bar. The desired amount of isobutylbenzene and acetyl chloride were added into the reactor. The reaction mixture was stirred under desired reaction temperature and time. After a certain reaction time, stirring was stopped, and a small aliquot of mixture was withdrawn from the reaction mixture and monitored by using GC with FID. The results are shown in Table 2.

The results of Table 2 show that the use of the acid only (triflic acid) as well as the combination of the acid with the ionic liquid provide surprisingly high conversions and selectivities despite the fact that a catalyst:acetyl chloride mole ratio of about 0.26:1 was used. Conversions and selectivities would be expected to be about an order of magnitude lower using the same conditions, but replacing the catalysts of Table 2 with existing catalysts such as HF and AlCl₃.

TABLE 2
Alkylation of isobutylbenzene reaction results using catalysts.

| | | Catalyst | | | | Product |
|---|---|---|---|---|---|---|
| | | Amount | T | Time | Conv. | Sel. |
| | Catalyst | (g) | (° C.) | (h) | (%) | (%) |
| 1 | [C₁im-SO₃H][TFES] | 0.2 | 25 | 3 | Nil | Nil |
| 2 | ([C₁im-SO₃H][TFES])₁₀-(TFMSA)₉₀ | 0.5 | 25 | 3 | >99.9 | 96.1 |
| 3 | ([C₁im-SO₃H][TFES])₁₀-(TFMSA)₉₀ | 0.2 | 25 | 3 | 76.9 | 96.0 |
| 4 | | 0.2 | 25 | 3 | 80.4 | 95.2 |
| 5 | | 0.2 | 25 | 3 | 72.8 | 95.4 |
| | | Average | | | 76.7 | 95.5 |
| | | Std dev | | | 3.8 | 0.4 |
| 6 | ([C₁im-SO₃H][TFES])₂₀-(TFMSA)₈₀ | 0.2 | 25 | 3 | 62.1 | 95.9 |
| 7 | ([C₁im-SO₃H][TFES])₅₀-(TFMSA)₅₀ | 0.2 | 25 | 3 | 52.7 | 96.0 |
| 8 | TFMSA | 0.2 | 25 | 3 | 74.7 | 95.3 |
| 9 | | 0.2 | 25 | 3 | 72.3 | 95.3 |
| 10 | | 0.2 | 25 | 3 | 67.6 | 95.6 |
| | | Average | | | 71.5 | 95.4 |
| | | Std dev | | | 3.6 | 0.2 |
| 11 | ([C₁im-SO₃H][TFES])₁₀-(TFESA)₉₀-(HMB)₁₀ | 0.2 | 25 | 3 | 60.8 | 95.2 |

Reaction condition: Catalyst (0.2 g), Isobutylbenzene (2.7 g; 20 mmol), acetyl chloride (0.4 g; 5 mmol), room temperature, 3 h.
Note:
TFMSA = Triflic acid; HMB = hexamethylbenzene.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A process for acylating an aromatic compound, the process comprising combining an aromatic compound, an acylating agent, and a catalyst composition under conditions to induce acylation of the aromatic compound with the acylating agent, the catalyst composition comprising
a sulfonic acid of formula R—SO₃H, wherein R is a linear alkyl group substituted with one or more halogen atoms, and wherein R has from 4 to 12 carbon atoms;
wherein the catalyst composition is free of a metal halide.

2. The process of claim 1, wherein the catalyst composition further comprises an ionic liquid and the ionic liquid is free of a metal halide.

3-5. (canceled)

6. The process of claim 1, wherein each of the one or more halogen atoms in the sulfonic acid are fluorine.

7-11. (canceled)

12. The process of claim 2, wherein ionic liquid comprises an imidazolium cation.

13. The process of claim 12, wherein the imidazolium cation has Formula B 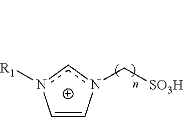
wherein R₁ is hydrogen or alkyl and n is 0, 3, 4, or 5.

14. The process of claim 13, wherein n is 0.

15. The process of claim 2, wherein the ionic liquid comprises [HCF₂CF₂SO₃]⁻ as an anion.

16. The process of claim 2, wherein the ionic liquid comprises an imidazolium as a cation and [HCF₂CF₂SO₃]⁻ as an anion.

17. The process of claim 1, wherein the aromatic compound is substituted and the acylating agent is an acyl halide, and the aromatic compound is acylated at its para position.

18. The process of claim 17, wherein the aromatic compound is isobutylbenzene and the acylating agent is acetyl chloride.

19. The process of claim 1, wherein a mole ratio of the catalyst composition to the acylating agent is no more than 0.5:1.

20. (canceled)

21. The process of claim 1, wherein R has from 4 to 9 carbon atoms.

22. The process of claim 1, wherein R has from 7 to 12 carbon atoms.

23. A process for acylating an aromatic compound, the process comprising combining an aromatic compound, an acylating agent, and a catalyst composition under conditions to induce acylation of the aromatic compound with the acylating agent, the catalyst composition comprising a sulfonic acid of formula R—SO₃H, wherein R is a linear alkyl group substituted with one or more halogen atoms, the catalyst composition further comprises an ionic liquid other than an imidazolium ionic liquid, and the ionic liquid does not comprise a metal halide.

24. The process of claim 23, wherein each of the one or more halogen atoms in the sulfonic acid are fluorine.

25. The process of claim 23, wherein R has from 1 to 12 carbon atoms.

26. The process of claim 23, wherein R has from 2 to 9 carbon atoms.

27. The process of claim 23, wherein the aromatic compound is substituted and the acylating agent is an acyl halide, and the aromatic compound is acylated at its para position.

28. The process of claim 27, wherein the aromatic compound is isobutylbenzene and the acylating agent is acetyl chloride.

29. The process of claim 23, wherein a mole ratio of the catalyst composition to the acylating agent is no more than 0.5:1.
